(19) United States
(12) Patent Application Publication
(10) Pub. No.: US 2021/0190803 A1
(43) Pub. Date: Jun. 24, 2021

(54) DEVICE AND METHOD FOR REVERSIBLY IMMOBILISING BIOMOLECULES

(71) Applicant: HOMBRECHTIKON SYSTEMS ENGINEERING AG, Hombrechtikon (CH)

(72) Inventors: Kai HASSLER, Luzern (CH); Konstantin LUTZE, Uerikon (CH); Harald QUINTEL, Steckborn (CH)

(21) Appl. No.: 16/761,314

(22) PCT Filed: Nov. 17, 2017
(86) PCT No.: PCT/EP2017/079622

§ 371 Date: May 4, 2020

(51) Int. Cl. G01N 35/00; B01L 3/00; C12N 15/10; B01L 3/02

(52) U.S. Cl. CPC G01N 35/0098; B01L 3/50255; C12N 15/1013; B01L 3/0268; B01L 3/5025; B01L 2400/0683; B01L 2300/049; B01L 2400/049; B01L 2300/0663; B01L 2400/043; B01L 2300/0829; B01L 2300/14; B01L 2400/0688

(57) ABSTRACT

A device for the reversible immobilization of biomolecules includes a container which can be filled with a liquid containing biomolecules and has an opening and a valve. The valve can be opened and closed by a closing mechanism for the controllable drainage of the liquid. Magnetic particles, to which the biomolecules can be immobilized can be arranged freely movable in the container. A magnet for fixing the magnetic particles in the container is arranged at the container, and the liquid can be removed from the container through the opening in the open state of the valve.

### CROSS-REFERENCE TO RELATED APPLICATION

This application is a U.S. National Stage application of International Application No. PCT/EP2017/079622, filed Nov. 17, 2017, the contents of which are hereby incorporated herein by reference.

### BACKGROUND

### Field of the Invention

The invention relates to a device for the reversible immobilization of biomolecules. The invention further relates to a method for the reversible immobilization of biomolecules and to an apparatus for the automated processing of biomolecules comprising a device for the reversible immobilization of biomolecules.

### Background Information

Many methods for the purification of DNA and other biomolecules are known in the state of the art. One type of purification is DNA extraction, in which the DNA is precipitated in a nonpolar environment. DNA can also be purified by centrifugation, e.g. after cell disruption, or by electrophoretic methods.

Biomolecules can also be synthesized and purified by immobilization on an insoluble carrier. Common substrates for immobilizing biomolecules are glass and other less common substrates such as gold, platinum, oxides, semiconductors and various polymer substrates.

“Magnetic bead-based clean-up” and “magnetic bead-based normalization” are widely spread methods for immobilization, purification and concentration adjustment of nucleic acids. Typical fields of application of these methods are sample preparation in the context of DNA sequencing or DNA detection (e.g. by PCR, polymerase chain reaction).

In the state of the art, the magnetic particles are typically held in the container by ring magnets which enclose a container. This allows a solution with impurities to be pipetted off, while the magnetic particles with the bonded biomolecules remain in the container.

The magnetic particles (magnetic beads) were developed in 1995 at the Whitehead Institute for the purification of PCR products. The magnetic particles are paramagnetic and can consist, for example, of polystyrene, which is coated with iron. Various molecules with carboxyl groups can then be attached to the iron. These carboxyl groups can reversibly bond DNA molecules. In doing so, the DNA molecules are immobilized.

Methods with magnetic particles usually comprise the following steps. First, the PCR products are bonded to the magnetic particles. Subsequently, the magnetic particles with the attached PCR products are separated from impurities (this step is realized e.g. by pipetting off the solution from the solid). The magnetic particles with the attached PCR products are then washed. After washing, the PCR products are eluted from the magnetic particles and transferred to a new plate.

### SUMMARY

In fully automated processes, the necessary reagents are automatically pipetted to the sample after the starting material has been introduced in an isolation process and are removed again by a pipette tip. The magnetic particle-bonded nucleic acids are collected at the bottom and at the edge of the cavities and, depending on the routine, again dissolved by optimized pipetting on and off. Finally, the DNA or RNA is eluted into separate vessels with lids for direct storage or further applications.

These steps therefore require repeated addition and removal of liquids or reagents. This is typically realized by pipetting with disposable pipette tips into microtiter plates (96 samples or more). These methods therefore have the great disadvantage that a large number of pipette tips are consumed, as they have to be changed after each step.

Furthermore, various dosing methods are known from the state of the art. For example, the I-DOT technology (“immediate Drop on Demand Technology”) of Dispendix, which is only a dispensing system. This system for liquid dispensing is based on a microtiter plate with so-called “wells”, which have openings of a few micrometers in diameter at the bottom. The liquid is held in the wells by capillary forces. A drop of precise volume, which is discharged through the lower openings of the wells, is formed by a well-defined pressure pulse from above onto a liquid-filled well. Thus, although precise amounts of liquid in the nanoliter range can be dispensed, a dispensing system does not offer the possibility of purifying biomolecules.

A dispensing device is also known from the U.S. Pat. No. 8,877,145 B2. In the device, a liquid is held by a capillary, which has a liquid reservoir. By applying a hydraulic pressure, the capillary forces are overcome, and a precise amount of fluid can be dispensed.

A device is known from U.S. Pat. No. 4,111,754 in which a plastic structure for surface enlargement is arranged in a capillary. In this capillary, the liquid is held by the capillary forces and antigens or antibodies can adhere to the plastic structure. In this way, the antigens or antibodies can be immobilized on the plastic surface. The impurities can then be removed by adding washing liquid. A disadvantage of this device is that the antigens and antibodies are bonded inside the capillary and cannot be ejected with the carrier material. The antigens and antibodies can only be eluted by dissolving them from the container, i.e. mobilizing them again. Furthermore, the surface to which the biomolecules are attached can only be adapted by changing the capillary, i.e. by changing the device, and during a reaction the carrier for the biomolecules cannot be moved for better mixing, which also increases the reaction time. In addition, the described device is not compatible with all purification protocols, which makes it difficult to integrate the device into existing workflows.

The main disadvantages of the state of the art are on the one hand that many pipette tips are consumed and on the other hand that the biomolecules are attached to stationary carrier materials. Thus, the methods known in the state of the art are slow, cost-intensive and not very efficient.

The object of the invention is therefore to provide a device for the immobilization of biomolecules by bonding the biomolecules to a solid surface, a method for the reversible immobilization and purification of biomolecules by bonding the biomolecules to a solid surface and an apparatus for the automated processing of biomolecules with a device for the immobilization of biomolecules, which avoid the adverse effects known from the state of the art.

The object is met by a device for the reversible immobilization of biomolecules as described herein, by a method for the reversible immobilization of biomolecules as described herein and by an apparatus for the automated processing of biomolecules comprising a device for the reversible immobilization as described herein.

Particularly advantageous embodiments of the invention are further described below.

According to the invention, a method for the reversible immobilization, in particular for the purification, of biomolecules is further proposed, carried out with a device for the reversible immobilization, in particular for the purification, of biomolecules. The method can comprise the following steps. Magnetic particles and a liquid, in particular a liquid with reagents, are arranged in a container. Biomolecules and reagents are bonded to the magnetic particles, in particular reversibly bonded. The magnetic particles are fixed with a magnet in the container. The liquid, in particular the liquid with impurities, is removed from the opening of the container by opening the valve, in particular for purification of the biomolecules. The biomolecules are dissolved from the magnetic particles, e.g. with a solvent. Subsequently, the dissolved biomolecules can be removed from the container by opening the valve.

Within the framework of the invention, the container can have a second opening. Liquid, for example, can be supplied or the valve can be controlled via this second opening. The second opening can be located on the opposite side of the container from the opening. The valve can be controlled via the second opening in such a way that a pressure on the liquid is regulated via the second opening.

For the reversible immobilization, in particular purification, with the magnetic particles, containers are used whose wells have one (or more) openings, preferably at the bottom, which are designed in such a way that they have a valve function or are controllable via a valve, so that it is possible to keep liquid in the well or empty the well through the openings, wherein the magnetic particles are held in the well of the container by the magnet. In addition, the biomolecules are reversibly bondable to the particles and the magnetic particles can have an enlarged surface compared to the container wall and can also be removed from the container together with the bonded biomolecules. Furthermore, the biomolecules can be selectively bonded to the surface of the magnetic particles so that only one type of biomolecule is bonded from a liquid.

The use of magnetic particles has a significant advantage that the magnetic particles can be easily fixed in the wells of the container by a magnet (e.g. permanent or electromagnet) or by a magnetic field, which allows an easy separation of the liquid. In addition, it is possible that the magnet is movably arranged on the container in such a way that the magnetic particles are freely movable in the container during a reaction step and are fixed in the container during a washing step by changing the magnet position. In particular, the magnet can be movable in such a way that the magnet is arranged in a first position on the container and fixes the magnetic particles and by moving the magnet to a second position on or around the container, the magnetic particles become movable.

Within the framework of this invention, the term biomolecule is understood to mean DNA, RNA, nucleic acids, proteins, start sequences for biomolecules, cells and cell components, monomers or other biologically relevant molecules.

Within the framework of embodiments of the invention, a washing step is a process step in which the liquid is discharged from the containers by actuating the valve and in which the impurities of magnetic particles with the attached biomolecules are thus separated. A washing step can also include washing with a washing solution (water or others).

Within the framework of embodiments of the invention, a reaction step is a process step in which the biomolecules bonded to the magnetic particles are converted, bonded to the particles or extended (chain extension, e.g. PCR “polymerase chain reaction”).

Within the framework of embodiments of the invention, reagents are understood to mean all compounds, molecules and liquids suitable for synthesis, purification and immobilization/mobilization. In particular, reagents can also be biomolecules and/or their monomers.

In the following, an impurity is generally a substance that is not fully reacted or bonded to the magnetic particles, the solvent, by-products and contaminants, as well as a mixture of two or more of the described above.

In particular, impurities can also be reagents or biomolecules.

Within the framework of embodiments of the invention, a liquid can be a solution, in particular a reaction mixture of biomolecules and/or reagents and/or impurities.

Within the framework of embodiments of the invention, purification is understood to mean the removal of impurities from the biomolecules bonded to the magnetic particles. In particular, purification can correspond to the removal of the liquid, especially the removal of a liquid after a washing step or the removal of the liquid between reaction steps. Within the framework of the invention, purification can also be understood as the normalization of biomolecules and the selection of biomolecules.

Within the framework of embodiments of the invention, a closing mechanism can be a mechanical and/or electrical and/or magnetic device for closing and opening the valve. However, it is also conceivable within the framework of the invention that the valve according to the invention is a capillary. In this embodiment, a closing mechanism could be a substance whose addition to the liquid changes the viscosity and/or the surface tension of this liquid. With such a closing mechanism/capillary combination, a change in pressure would correspond to the reduction in surface tension and/or viscosity.

Within the framework of embodiments of the invention, a pressure changer can be a device for generating pressure (liquid and/or air pressure), such as a pump, a blower or a punch. A pressure changer can also be a device that manipulates a film in such a way that a pressure can be exerted to the liquid. Furthermore, a pressure changer can be a device for pulling a container and a collecting device apart in order to release excess pressure that retains the liquid.

Within the framework of embodiments of the invention, the retention force of the valve can be the capillary force of a capillary, the negative pressure generated by a film and generally a negative pressure, the surface tension and/or the viscosity of a liquid, an excess pressure, in particular an excess pressure generated by a collecting container, a fluid barrier generated by a filter, or a magnetic or mechanical force of a closing mechanism.

Within the framework of embodiments of the invention, immobilization is understood to mean the bonding, in particular the reversible bonding of the biomolecules to the magnetic particles.

In the following, a magnetic particle (also called a “magnetic bead”) can generally be a particle in the micrometer or millimeter range. Furthermore, a magnetic particle can be porous. in the following, a biomolecule can generally be bonded to the surface of magnetic particles via thiol groups and/or amino groups and/or hydroxy groups and/or carboxyl groups and/or carbonyl groups and/or ester groups and/or nitrile groups and/or amine groups and/or any other functional groups.

Within the framework of embodiments of the invention, a magnetic particle can be a coated nickel particle or any other ferro- or paramagnetic particle. Magnetic particles typically have a diameter of about 1 micrometer. Within the framework of the invention, approximately 1 micrometer is understood to mean 0.5 to 1.5 micrometer, in particular 0.7 to 1.3 micrometer, especially 0.9 to 1.1 micrometer.

In the following, a valve can generally also be a pressure valve, a flow valve or a non-return valve, particularly preferably a capillary and/or a filter and/or a film and/or a collecting container and/or a magnetically controlled valve.

Within the framework of embodiments of the invention, a magnet can be a permanent magnet and/or an electromagnet and/or a superconductor and/or a ferromagnet and/or a paramagnet. In particular, a magnet can be a device that exerts a magnetic force.

Within the framework of the invention, a measuring instrument can be a luminescence and absorption measuring instrument or a fluorescence measuring instrument or a UV-Vis measuring instrument or a nanopore-based measuring instrument.

The advantages of the device according to the invention and method according to the invention are:

drastic reduction of pipette tip consumption

reduction of the process time (since pipetting steps are eliminated)

an instrument based on this method can be realized in a comparatively space-saving way

efficient and cost-effective

easy to automate

also for devices of reduced size

allows easy modification of existing machines

biomolecules can be removed immobilized from the sample container and further processed

biomolecules can be selectively bonded

a much larger surface area is produced by using particles

processing of smaller volumes

no residual volumes

the device can be easily integrated into existing (manual, semi-automated or automated) workflows (can build on standard procedures for DNA purification)

compatible with established particle-based purification protocols, so that the device can be easily integrated into existing workflows

In practice, the closing mechanism can he a pressure changer, wherein a pressure on the liquid can be changed by the pressure changer in such a way that a retention force of the valve can be overcome by the pressure. In this way, the valve can be opened. Controlling the pressure on the liquid is important to empty the well if necessary. The pressure can be controlled by a pressure chamber which is connected to the upper part of the wells or the container (the upper part being the part through which pressure can be applied to the liquid). When using a multi-well plate, in particular a microtiter plate, individual areas or wells can be independently applied with pressure by independent pressure chambers (e.g. one pressure chamber per well or per area of the multi-well plate). For this purpose, a pressure chamber arrangement with independent pressure chambers can be connected to the upper part of the well or container. The pressure difference can also be created by creating a negative pressure on the outside of the opening. In order to control the pressure difference between the inside and outside of the well or container, the upper part of the well or container and/or the lower opening can be closable. Reversible closing is also conceivable for longer storage of samples or reagents in the container (possibly reversible closing to make a multiwell plate, in particular a microtiter plate, PCR-compatible).

When using a pressure changer, the opening of the valve corresponds to an increase in pressure on the liquid or a negative pressure created which acts on the liquid at the opening of the container. The valve is always closed when no liquid can be removed from the container through the opening (only if there is still liquid in the container). For example, a pressure changer can work by the following principles: hydrostatic, capillary pressure, centrifugal force, gas pressure.

In an embodiment of the invention, the closing mechanism can be a hydrostatic pressure changer, wherein by the hydrostatic pressure changer a hydrostatic pressure of the liquid can be increased by the addition of liquid into the container in such a way that a retention force of the valve can be overcome by the hydrostatic pressure, and thus the valve can be opened. This makes it possible to remove a part of the liquid from the container by adding new liquid, i.e. increasing the filling level of the container. Thus, a hydrostatic pressure changer could be a supply device for a liquid (for example a washing liquid for a washing step).

In practice, a polarity and/or viscosity and/or surface tension of the liquid in the container can be changeable by the closing mechanism, so that a retention force of the valve can be overcome and thus the valve can be opened. The polarity and/or viscosity and/or surface tension of the liquid can be changed, for example by adding other liquids or substances, or by changing the pH value. Thus, the closing mechanism could be designed as a supply device for a substance (for example surfactants for surface tension; non-polar or polar liquids; solids) or a liquid. For a change in viscosity, a heating device as a closing mechanism would also be possible.

In an embodiment of the invention, the pressure changer can change the air pressure above the liquid and/or at the opening, in particular an opening arranged at the bottom of the container. The creation of a negative pressure at the opening can lead to the drainage of the liquid. In addition, an increase in air pressure above the liquid can lead to the drainage of the liquid. Thus, the valve would be opened by creating the negative pressure at the opening and by increasing the air pressure above the liquid. The term “air pressure above the liquid” means the air pressure which also acts on the liquid in such a way that the liquid can be removed from the container.

In an embodiment of the invention, the valve of the device can be arranged at the opening. The valve can also be the opening, e.g. if the valve is a capillary, the opening of the capillary is also the opening for discharging the liquid. In particular, the valve and/or the opening can he arranged at the bottom of the container.

In practice, a well in the device can comprise several valves and/or openings. Thus, the openings could also act as a kind of screen through which the magnetic particles cannot pass, but the liquid can drain. Such a construction is also possible if there are several capillaries at one well as valves.

In an embodiment of the invention, the valve of the device can be designed as a capillary or as a filter or as a film or as a collecting container.

If the valve function is realized in such a way that the lower opening is designed as a thin capillary, the capillary pressure is sufficient to prevent a spontaneous emptying of the cavities. The liquid can now be removed by applying a pressure pulse (by the pressure changer) to the liquid from above so that the liquid is removed through the opening (opening the valve). If the valve is designed as a filter, the liquid is retained by the fluid barrier of the filter material. Here, the liquid can also be removed here by applying a pressure pulse (by the pressure changer) to the liquid from above so that the liquid is pressed through the filter (opening the valve) and removed through the opening. If the valve is a film, the film can be arranged above the container in such a way that a gas volume is enclosed between the film and the liquid. Now, by manipulating the film (e.g. by moving the film by a pressure generated by the pressure changer) the gas volume between liquid and film can be compressed in such a way that a pressure is exerted on the liquid, which presses the liquid out of the opening (opening the valve).

In practice, the opening of the device can be closable with a bead which is floatable on the liquid. Thus, there is the possibility to empty the liquid via the opening and then close the opening of the well.

In an embodiment of the invention, the container of the device is a multiwell plate, in particular a microtiter plate, with wells.

In an embodiment of the invention, the pressure changer of the device can be a pressure chamber arrangement so that each well can be individually applied with pressure.

A measuring instrument can be arranged on the valve or in the container so that a measurement can be carried out on the hanging drop or with the liquid in the container.

In an embodiment of the invention, the device can comprise a mixer. The mixer can be a modifiable magnetic field and/or a magnetically movable solid body. In this case, a magnetically movable solid body can be a stirring rod and/or magnetic stirrer, which is set in motion by a magnetic field. When using magnetic particles, a movement of the magnetic particles can be caused by a modifiable magnetic field, which also causes mixing.

In practice, devices can also be connected in series.

In practice, the device and method can be used for post ligation purification. According to the invention, a method for the reversible immobilization, in particular for the purification, of biomolecules is further proposed, carried out with a device for the reversible immobilization, in particular for the purification, of biomolecules. The method can comprise the following steps. Magnetic particles and a liquid with reagents are arranged in a container. Biomolecules or reagents for the synthesis of biomolecules are bonded to the magnetic particles, in particular reversibly bonded. The magnetic particles are fixed in the container with a magnet. The liquid with impurities is removed from the opening of the container by opening the valve to purify the biomolecules. The biomolecules are dissolved from the magnetic particles, e.g. with a solvent. Subsequently, the dissolved biomolecules can be removed from the container by opening the valve.

Of course, the method can comprise multiple steps in which liquids must be added and discharged and impurities separated or in which the biomolecules are dissolved from the magnetic particles. in this way, the purified biomolecules can be dispensed by discharging them through the opening of the device after dissolving them from the magnetic particles.

If the magnetic particles are fixed in the container with a magnet, the liquid can subsequently be removed by changing the pressure (depending on the valve type). Such a procedure can be useful after completion of a reaction step, either to carry out a further reaction step or to separate the impurities in a washing step.

According to the invention, an apparatus for the automated processing of biomolecules with a device for the reversible immobilization, in particular for the purification of biomolecules, is further proposed.

### BRIEF DESCRIPTION OF THE DRAWINGS

The invention will be explained in more detail hereinafter with reference to the drawings.

FIG. 1 is a schematic representation of a device for the reversible immobilization and purification of biomolecules.

FIG. 2 is a schematic representation of a further embodiment of a device for the reversible immobilization and purification of biomolecules.

FIG. 3 is a schematic representation of a further embodiment of a device for the reversible immobilization and purification of biomolecules.

FIG. 4 is a first embodiment of a valve.

FIG. 5 is a second embodiment of a valve.

FIG. 6 is a third embodiment of a valve.

FIG. 7 is a schematic representation of a further embodiment of a device for the reversible immobilization and purification of biomolecules.

### DETAILED DESCRIPTION OF THE EMBODIMENTS

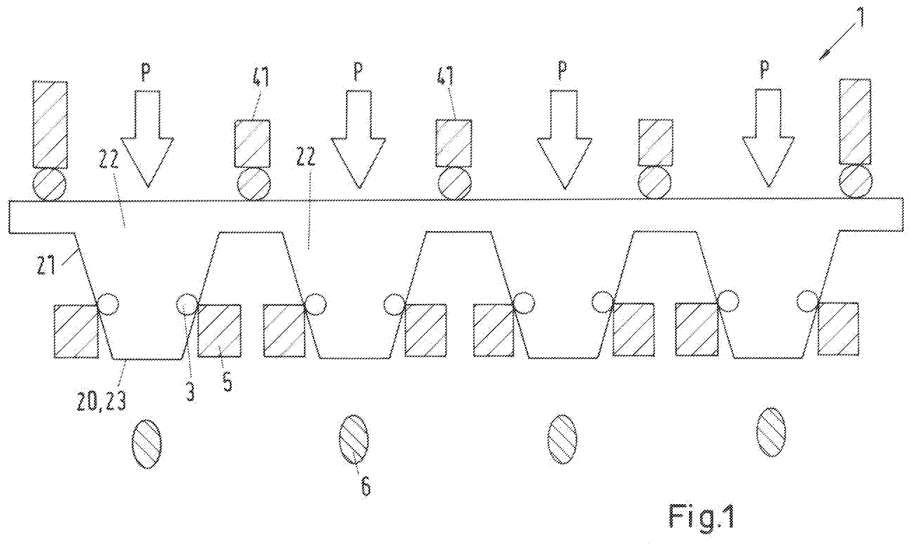

FIG. 1 shows a schematic representation of a device 1 for the reversible immobilization and purification of biomolecules. In this embodiment, the container is designed as multiwell plate 21. The wells 22 of the multiwell plate 21 can be filled with a liquid 6. In this embodiment, the magnetic particles 3 are arranged in the wells 22 of the multiwell plate 21 and are designed as a collection of magnetic particles. In a method for processing biomolecules, a liquid 6 with the biomolecules to be processed together with the reagents required for this purpose would be located in the wells 22 of the multiwell plate 21. The biomolecules, which are located in the liquid 6, can be reversibly attached to the magnetic particles 3 (i.e. they can be immobilized). The desired biomolecules can be selectively bonded to the magnetic particles. The non-bonded impurities are then removed via the opening. In addition, the biomolecules can be extended e.g. at the surface of the magnetic particles 3 (e.g. by PCR). After a completed reaction, any impurities which have been formed during the reaction or which have not completely reacted, and which are present in the liquid 6 must be removed. For this purpose, a pressure p generated by a pressure changer, which here is designed as a pressure chamber arrangement 41 (here device generating a pressure p), can overcome the retention force of the valve 20 by exerting a pressure on the liquid 6 (not shown here) located in the wells. In this way, the liquid 6 can be removed from the multi well plate 21, while the biomolecules remain on the surface of the magnetic particles 3. The magnetic particles are held in the well 22 of the multiwell plate 21 by a magnet 5.

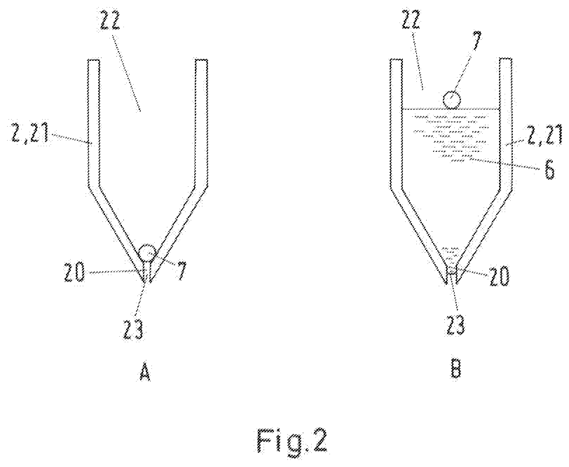

FIG. 2 shows a schematic representation of a further embodiment of a device 1 for the reversible immobilization and purification of biomolecules. in this device 1, a floatable bead 7 is arranged in the container 2, 21 in the well 22. In condition A, in which there is no liquid 6 in the container 2, 21, the floating bead 7 closes the opening 23 and the valve 20. The valve 20 can be, for example, a capillary in which the liquid 6 is held by the capillary forces.

In the embodiment in which the container 2, 21 is designed as a multiwell plate 21, and in which several wells 22 are arranged next to each other, a pressure drop can thus be prevented when emptying the wells 22 by applying a pressure p (not shown here) generated by the pressure changer (here the device generating a pressure p). The pressure drop occurs when one well of the multiwell plate 21 is already empty, i.e. is in condition A, while other wells 22 of the multiwell plate 21 are still filled with liquid 6, i.e. are in condition B. The pressure drop can be prevented by closing the opening 23 of a well 22, which is in condition A, by the floatable bead 7.

In condition B, in which the well 22 is filled with liquid, the floatable bead 7 floats on the surface of the liquid 6 and thus allows the liquid 6 to be removed from the opening 23 by applying a pressure p (not shown here). In condition B, the liquid 6 is held by the valve 20 in the well 22 of the container 2, 21 and cannot drain through the opening 23. The liquid 6 can drain from the opening 23 only when the valve 20 is opened.

A floatable bead can he used, for example, in a device as shown in FIG. 1.

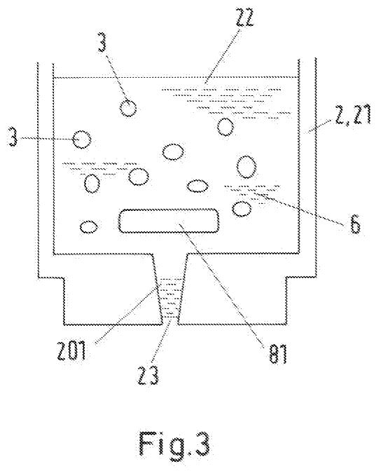

FIG. 3 shows a schematic representation of a further embodiment of a device for the reversible immobilization and purification of biomolecules. In this embodiment, a liquid 6 with magnetic particles 3 is located in the container 2, 21. The liquid 6 is retained by a valve 20 in the form of a capillary 201. Furthermore, a stirring rod 81 is located in the well 22 of the container 2, 21, This stirring rod 81 is suitable for setting the liquid 6 in motion in such a way that the liquid 6 is thoroughly mixed during a reaction step. During a washing step, the liquid 6 can drain faster by applying a pressure p (not shown here) if the liquid 6 is set in motion by the stirring rod 81.

Of course, the stirring rod 81 shown in FIG. 3 can be combined with any valve 20 and the stirring rod 81 can also be designed as another magnetically movable solid body.

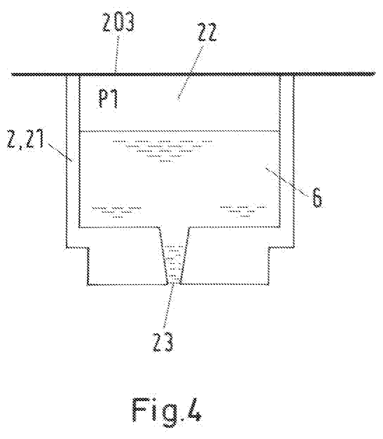

FIG. 4 shows a first embodiment of a valve. In this embodiment, the valve of the container 2, 21, is designed as a film 203. The opening 23 need not be a capillary but can simply be designed as a channel. Due to the film 203, the liquid 6 cannot drain through the opening 23 from the well 22 of the container 2, 21, because the liquid is held in the container by a negative pressure. Only when the film 203 is moved, when the gas volume between film and liquid 6 is compressed, i.e. when a pressure P3 is applied to the liquid, the liquid 6 can drain through the opening 23. The film 203 could be moved by a pressure changer in such a way that the film 203 causes a lowering of the film 203 in the direction of the liquid 6 by a pressure (not shown here) on the film from the side away from the liquid. In a method according to the invention, the magnetic particles 3 could be held in the well 22 by a magnet 5 in a washing step, while the liquid 6 together with impurities could drain when moving the film 203 (magnetic particles 3 and magnet 5 see FIG. 1). Of course, a valve according to FIG. 4 can be combined with a device 1 according to FIG. 1, as well as with a floatable bead 7 according to FIG. 2 and a stirring rod 81 according to FIG. 3.

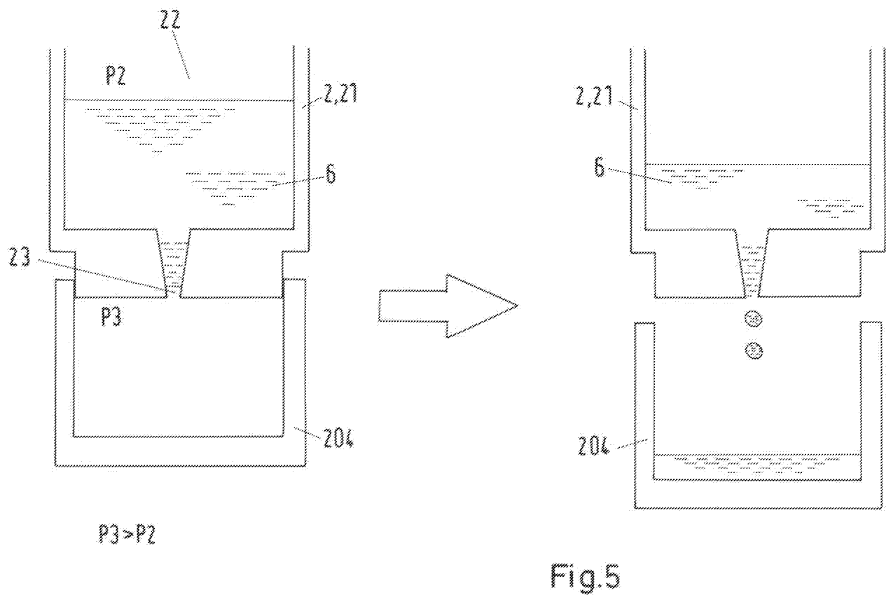

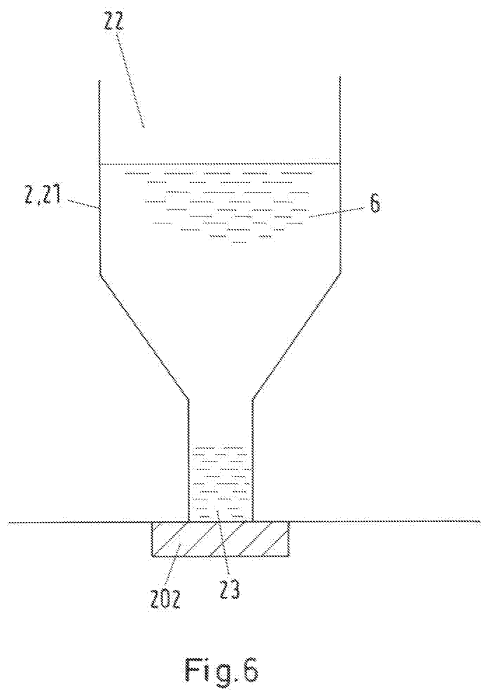

FIG. 5 shows a second embodiment of a valve. In this embodiment, the valve of the container 2, 21 is designed as a collecting container 204. An excess pressure P1 is generated in the collecting container 204 in such a way that the liquid 6 cannot drain of the well 22 of the container 2, 21 through the opening 23. Only when the container 2, 21 and the collecting container 204 are pulled apart, when the excess pressure P1 adapts to the ambient pressure P2, the liquid 6 can drain through the opening 23. In a method according to the invention, the magnetic particles 3 could be held in the well 22 by a magnet 5 in a washing step, while the liquid 6 together with impurities can drain when the container 2, 21 and the collection container 204 are pulled apart (magnetic particles 3 and magnet 5 see FIG. 1). In this embodiment, a pressure changer would correspond to a device for pulling apart the container 2, 21 and the collecting container 204, as this changes the excess pressure P1 to the ambient pressure P2, allowing the liquid 6 to drain. Of course, a valve according to FIG. 5 can be combined with a device 1 according to FIG. 1, as well as with a stirring rod 81 according to FIG. 3. In addition, it is possible that a pressure change is implied differently. For example, the pressure change can be caused by a closable opening, which is arranged on the collecting container 204. FIG. 6 shows a third embodiment of a valve. in the case of the container 2, 21, the valve is designed as a filter 202. The liquid 6 is retained by the filter 202, so that the liquid 6 cannot drain through the opening 23 from the well 22 of the container 2, 21. Only when a pressure P (not shown here) is generated by a pressure changer (here rather a pressure generator), which applies the liquid 6 in such a way that the liquid 6 is pressed through the filter 202, the liquid 6 can drain through the opening 23. In a method according to the invention, the magnetic particles 3 could be held in the well 22 by a magnet 5 in a washing step, while the liquid 6 together with impurities can drain when applying with pressure. In this embodiment, a pressure changer would correspond to a device for generating pressure, since this overcomes the retention force of the 202 filter, allowing the liquid 6 to drain. Of course, a valve according to FIG. 6 can be combined with a device 1 according to FIG. 1, as well as with a stirring rod 81 according to FIG. 3.

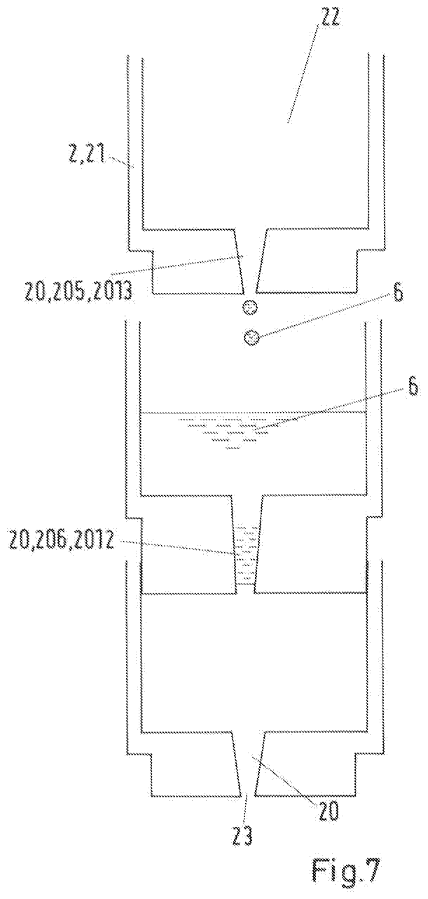

FIG. 7 shows a schematic representation of a further embodiment of a device for the reversible immobilization and purification of biomolecules. This embodiment shows a series connection of several devices. In this way, a liquid 6 can be transferred from an upper container 2, 21 to a lower container 2, 21 by actuating the valve 20 to transfer the liquid from one opening 23 to the next container 2, 21. The valves 20 of the different containers can all be the same or all different or partially different, For example, a first valve 205 could be a capillary 201, while a second valve 206 is a filter. But it would also be conceivable that a first valve 205 is a first capillary 2013, while a second valve 206 is a second capillary 2012. Thus, the first and second capillaries 2012, 2013 can be of different length and/or thickness, whereby a different residence time of the liquid 6 is achieved in each container 2, 21, Of course, a series connection according to FIG. 7 can be combined with a device 1 according to FIG. 1, as well as a floatable bead 7 according to FIG. 2 and a stirring rod 81 according to FIG. 3. In addition, with a series connection, various process steps can be carried out at each level of the device,

1. A device for the reversible immobilization of biomolecules, wherein the device comprising: comprises
a container configured to be filled with a liquid containing biomolecules and having an opening and a valve, the valve configured to be opened and closed by a closing mechanism to enable controllable drainage of the liquid;
wherein
magnetic particles, to which the biomolecules are capable of being immobilized, the magnetic particles arranged to be freely movable in the container; and
a magnet for fixing the magnetic particles in the container is arranged at the container, the liquid being removable from the container through the opening in an open state of the valve.

2. The device according to claim 1, wherein the closing mechanism is a pressure changer, and the pressure changer is configured to change a pressure on the liquid such that a retention force of the valve is capable of being overcome by the pressure to open the valve.

3. The device according to claim 1, wherein the closing mechanism is configured to change a polarity or a viscosity or a surface tension of the liquid in the container, so that a retention force of the valve is capable of being overcome to open the valve.

4. The device according to claim 2, wherein the pressure changer is a hydrostatic pressure changer, and the hydrostatic pressure changer is configured to increase a hydrostatic pressure of the liquid by adding additional liquid into the container such that a retention force of the valve is capable of being overcome by the hydrostatic pressure to open the valve.

5. The device according to claim 2, wherein the pressure changer is configured to change the air pressure above the liquid or at the opening.

6. The device according to claim 1, wherein the valve is arranged at the opening.

7. The device according to claim 1, wherein the valve is a capillary or filter or a film or a collecting container.

8. The device according to claim 1, wherein the opening is configured to be closed with a bead which is floatable on the liquid.

9. The device according to claim 1, further comprising a measuring instrument arranged at the opening or in the container, and being configured to carry out a measurement on a drop hanging at the opening or in the container, respectively.

10. The device according to claim 1, wherein the device comprises a mixer.

11. The device according to claim 10, wherein the mixer is a modifiable magnetic field or a magnetically movable solid body.

12. The device according to claim 1, wherein the container is a multiwell plate.

13. The device according to claim 12, wherein the wells comprise a plurality of valves or openings.

14. The device according to claim 13, wherein the closing mechanism is a pressure changer, and the pressure changer is a pressure chamber arrangement so that each well of the wells is configured to be individually applied with pressure.

15. The device according to claim 1, wherein the device is one of a plurality of devices connected in series.

16. A method for the reversible immobilization of biomolecules, the method comprising:
operating the device according to claim 1.

17. A method for the reversible immobilization of biomolecules, the method comprising:
arranging magnetic particles and a liquid containing biomolecules in a container;
bonding of the biomolecules to the magnetic particles;
fixing the magnetic particles with a magnet in the container;
removing the liquid from an opening of the container by opening a valve;
dissolving the biomolecules from the magnetic particles;
removing the dissolved biomolecules by opening the valve.

18. An apparatus for the automated processing of biomolecules comprising:
a device according to claim 1.

19. The device according to claim 1, wherein the biomolecules are capable of being reversibly immobilized by the the magnetic particles.

20. The device according to claim 12, wherein the container is a microtiter plate.
